(19) United States
(12) Patent Application Publication
(10) Pub. No.: US 2025/0134531 A1
(43) Pub. Date: May 1, 2025

(54) ARTICULATING SURGICAL CLAMP

(71) Applicant: DRACO Startup Studio, LLC, Sioux Falls, SD (US)

(72) Inventors: Sujit Vijay Sakpal, Sioux Falls, SD (US); Eric Sandhurst, Sioux Falls, SD (US); Jevin Meyerink, Thousand Oaks, CA (US)

(21) Appl. No.: 18/932,716

(22) Filed: Oct. 31, 2024

Related U.S. Application Data
(60) Provisional application No. 63/594,468, filed on Oct. 31, 2023.

(51) Int. Cl. A61B 17/122; A61B 17/28

(52) U.S. Cl. CPC A61B 17/122; A61B 17/282; A61B 2017/2808

(57) ABSTRACT

An articulating clamp for use during surgical procedures. An articulating surgical clamp for temporarily holding, clamping, and positioning patient tissue, for example, a vascular clamp for occluding a vessel. The articulating surgical clamp facilitates adjustment of the shape of the jaws of the clamp, for example, to improve vessel occlusion during surgical procedures. A method of temporarily occluding a blood vessel includes providing an articulating clamp as described herein, configuring the shape of the jaws of the clamp, and positioning the clamp to occlude a blood vessel.

### CROSS-REFERENCE TO RELATED APPLICATIONS

This application claims priority to U.S. Provisional Application No. 63/594,468, filed Oct. 31, 2023, which is hereby incorporated herein by reference in its entirety.

### FIELD

The various embodiments herein relate to surgical instruments and related methods of use. More particularly, the various embodiments herein relate to an articulable clamp (e.g., an articulable vascular clamp) that may be modified by a user during a procedure to customize the shape of the jaw portion of the clamp for a particular application.

### BACKGROUND

A surgical clamp or clamping instrument is a device that may be used in a number of surgical procedures for the temporary holding, clamping, and/or positioning of tissue, such as during the occlusion of blood vessels. For example, the Debakey vascular clamp is a known, ergonomically-designed clamping instrument. A vascular clamp may be used to help a surgeon occlude blood flow in cardiothoracic vessels to thereby prevent hemorrhage. A ratchet lock mechanism of the clamping instrument is often used to hold the device in a clamped position during longer procedures.

With known technologies, surgeons currently must adapt and choose from a variety of clamps to address variations in surgical anatomy. That is, surgeons across multiple fields typically use a “fixed” shape clamp design. During a surgical procedure, the surgeon is constantly evaluating the anatomy and surgical pathology that is unique to a given patient, which may require them to frequently change tools during an operation until one is found that best accomplishes the task at hand. Additionally, conventional surgical clamps are typically manufactured as single-unit devices, where the handle and clamp body are of a unitary design (e.g., permanently affixed to one another), limiting their adaptability in diverse surgical environments. Different procedures may require clamps with unique jaw profiles, or handles with certain mechanical or ergonomic properties, necessitating the use of multiple clamps during the same or different surgeries. This can increase operational costs, time, sterilization needs, and the volume of equipment present in operating rooms.

Accordingly, there is a need in the art for an improved surgical clamp that better addresses the needs of surgeons.

### BRIEF SUMMARY

Discussed herein are several embodiments of an improved clamp device for use during surgical procedures.

Some embodiments of this disclosure describe an adaptive clamp design that utilizes innovative articulating hinges and/or hinged segments to improve surgical precision by giving surgeons control over the tool's shape throughout the operation. The articulating clamp of this disclosure is designed with challenging surgical situations in mind and with the goal of addressing the limitations of traditional fixed clamps. By allowing users (surgeons) to rapidly adapt the tool to unique situations, both the number of instruments and the need for excessive and/or frequent instrument exchanges would be reduced. Thereby, we aim to introduce a new era of surgical excellence with enhanced adaptability and improved ergonomics that empowers surgeons to perform intricate procedures with confidence using our tool.

In some embodiments, a clamp includes a jaw portion for sealing a vessel, or for occlusion of a segment of a hollow organ such as the gastrointestinal tract. The jaw portion may comprise a number of articulating segments, each articulating segment configured to be releasably positionable with respect to an adjacent articulating segment and/or to the handle portion of the clamp itself. In some embodiments, the articulating segments may be adjusted to a number of defined angles to enable forming a desired overall shape of the jaw portion to thereby improve the functionality of the clamp for a particular use, e.g., for a particular anatomical shape for vessel sealing applications, for example.

In some embodiments, a button may be associated with one or more of the plurality of articulating segments. A button press (e.g., downward force on an outer surface of a button) may enable the articulation (e.g., angular relationship) of adjacent articulating segments with respect to each other. For example, a button may be configured to releasably engage and disengage from a number of pre-defined detent positions. The detent positions may correspond to a number of defined or pre-determined angles, according to some embodiments. Pressing on an outer surface or portion of such a button may push (e.g., inwardly) against a shaped paddle, moving the shaped paddle against a bias force; the bias force may be provided by a spring element in some embodiments. The button press may thereby disengage or “clear” portions of the shaped paddle from a corresponding shaped opening in a main body of the articulating segment. Once cleared, the adjacent articulating segment may be positioned (e.g., pivoted) to a new or different angle or detent position such that a subsequent release of the button press will re-engage the shaped paddle in a new angular position within the shaped opening of the main body, for example. That is, releasing the button press pressure will allow the spring bias force to move the paddle back into a new angular position within the shaped opening of the main body. The shape of the shaped opening may be a “star” shape, or an “asterisk” shape, or some other shape configured to facilitate a positive engagement or seating of the paddle in a number of defined detent positions.

In some embodiments, the shaped opening may have a star or asterisk pattern, with “arms” of the opening extending at 45 degree increments outwardly from the center of the star or asterisk opening. Thus, a paddle with four arms extending outwardly at right angles (90 degrees) may be rotated at 45 degree increments to a series of new angular positions within the corresponding shaped opening of the adjacent segment's main body. This may, for example, enable positioning an adjacent articulating segment at one of five (or more) possible angular orientations: 90, 45, 0, −45, and −90 degrees, for example, relative to each other. Of course, other angular increments are contemplated by this disclosure and may be used to achieve different levels of angular customization. For example, 30-degree increments might be used instead, which could enable 7 or more possible angular adjustments or orientations: 90, 60, 30, 0, −30, −60, and −90 degrees, for example, relative to each other. Fewer or greater numbers of angular adjustments may be accomplished if and as needed.

In some embodiments, a single-button may be used to adjust the angular disposition of adjacent articulating segments of the clamp. That is, each articulating segment may be angularly adjusted relative to an adjacent articulating segment via a single button operably engaged between them.

In some other embodiments, a two-button design may be used to adjust the angular disposition of adjacent articulating segments of the clamp. In such a design, each articulating segment may be angularly adjusted relative to an adjacent articulating segment via a link member disposed between the adjacent articulating segments operably coupled to each articulating segment by a separate button associated with each of the two adjacent segments. One or both of the buttons between each pair of adjacent segments may be selectively released and re-engaged to facilitate additional levels of customization in forming the overall shape of the jaw portion of a surgical clamp, according to some embodiments.

In some other embodiments, a surgical clamp may include a detachable handle, allowing various jaw portions and/or clamp body configurations to be securely attached to a number of different handle designs, for example. Such an embodiment may provide a modular aspect including releasably coupleable handle and/or jaw portions that may facilitate forming a more suitable combination of the handle and jaw portions. Such a modular feature may give users flexibility to customize their instruments to meet the specific needs of the procedure and patient, while maintaining operational safety, ease of use, and functionality.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF DRAWINGS

FIG. 1A is a side view of a closed articulating surgical clamp for use during surgical procedures according to some embodiments of this disclosure;

FIG. 1B is a top perspective view of a closed articulating surgical clamp for use during surgical procedures according to some embodiments of this disclosure;

FIG. 1C is a top view of a closed articulating surgical clamp for use during surgical procedures according to some embodiments of this disclosure;

FIG. 1D is a top perspective view of an articulating surgical clamp with a jaw portion in a partially open position according to some embodiments of this disclosure;

FIG. 2A is a top perspective view of a closed articulating surgical clamp with a jaw portion formed in an articulated shape according to some embodiments of this disclosure;

FIG. 2B is a top perspective view of an articulating surgical clamp with a jaw portion formed in an articulated shape and partially open according to some embodiments of this disclosure;

FIG. 2C is a top view of an articulating surgical clamp with a jaw portion formed in an articulated shape according to some embodiments of this disclosure;

FIG. 3 is an exploded perspective view of portions of an articulating surgical clamp according to some embodiments of this disclosure;

FIG. 4A is an enlarged exploded perspective view of an articulatable segment of a jaw portion of an articulating surgical clamp according to some embodiments of this disclosure;

FIG. 4B is an enlarged top view of a main body of an articulatable segment of a jaw portion of an articulating surgical clamp according to some embodiments of this disclosure;

FIG. 4C is an enlarged top perspective view of an end body of an articulatable segment of a jaw portion of an articulating surgical clamp according to some embodiments of this disclosure;

FIG. 5A is a side view of a closed articulating surgical clamp for use during surgical procedures according to some embodiments of this disclosure;

FIG. 5B is a top perspective view of a closed articulating surgical clamp for use during surgical procedures according to some embodiments of this disclosure;

FIG. 5C is a top view of a closed articulating surgical clamp for use during surgical procedures according to some embodiments of this disclosure;

FIG. 5D is a top perspective view of an articulating surgical clamp with a jaw portion in a partially open position according to some embodiments of this disclosure;

FIG. 6A is a top perspective view of a closed articulating surgical clamp with a jaw portion formed in an articulated shape according to some embodiments of this disclosure;

FIG. 6B is a top perspective view of an articulating surgical clamp with a jaw portion formed in an articulated shape and partially open according to some embodiments of this disclosure;

FIG. 6C is a top view of an articulating surgical clamp with a jaw portion formed in an articulated shape according to some embodiments of this disclosure;

FIG. 7 is an exploded perspective view of portions of an articulating surgical clamp according to some embodiments of this disclosure;

FIG. 8A is an enlarged exploded perspective view of an articulatable segment of a jaw portion of an articulating surgical clamp according to some embodiments of this disclosure;

FIG. 8B is an enlarged top view of a main body of an articulatable segment of a jaw portion of an articulating surgical clamp according to some embodiments of this disclosure;

FIG. 8C is an enlarged top perspective view of an end body of an articulatable segment of a jaw portion of an articulating surgical clamp according to some embodiments of this disclosure;

FIG. 9A is an enlarged top view of a jaw portion of a closed articulating surgical clamp according to some embodiments of this disclosure;

FIG. 9B is an enlarged top oblique/rotated view of a jaw portion of a closed articulating surgical clamp according to some embodiments of this disclosure;

FIG. 9C is an enlarged cross-sectional view of a jaw portion of a closed articulating surgical clamp according to some embodiments of this disclosure;

FIG. 10A is an enlarged top view of a jaw portion of a closed articulating surgical clamp according to some embodiments of this disclosure;

FIG. 10B is an enlarged top oblique/rotated view of a jaw portion of a closed articulating surgical clamp according to some embodiments of this disclosure;

FIG. 10C is an enlarged cross-sectional view of a jaw portion of a closed articulating surgical clamp according to some embodiments of this disclosure;

FIG. 11A is perspective view showing portions of an articulating clamp having a handle configured to be releasably engaged with a jaw portion, according to some embodiments of this disclosure;

FIG. 11B is an exploded perspective view showing portions of the exemplary articulating clamp of FIG. 11A according to some embodiments of this disclosure;

FIG. 11C is a side view showing portions of the exemplary articulating clamp of FIG. 11A, according to some embodiments of this disclosure;

FIG. 11D is an exploded side view showing portions of the exemplary articulating clamp of FIG. 11A, according to some embodiments of this disclosure;

FIG. 11E is a top view showing portions of the exemplary articulating clamp of FIG. 11A, according to some embodiments of this disclosure; and

FIG. 11F is an exploded top view showing portions of the exemplary articulating clamp of FIG. 11A, according to some embodiments of this disclosure.

### DETAILED DESCRIPTION

Disclosed herein are embodiments and features of an articulating mechanism that enables a surgeon to adjust the angle and/or position of multiple articulatable segments relative to each other along a jaw portion of a surgical clamp, such as a vascular clamp. When used to clamp a blood vessel, this adaptable design may facilitate vessel occlusion and can provide enhanced adaptability “on the fly.” That is, a surgeon may choose to change or adapt the shape of the jaw portion of the articulating clamp upon seeing a shape or pattern or orientation of the tissue, vessel, organ, etc., that is specific or unique to a particular patient's anatomy without needing to find and use a different clamp, for example. Embodiments disclosed here include a customizable, modular clamping device. Surgeons can pre-set specific clamp configurations tailored to variant anatomy and/or for different procedures using an assembly of modular articulatable segments to achieve desired angles and/or overall shapes for achieving optimal clamping of tissue, vessels, organs, and/or other anatomical structures. An articulating clamp according to embodiments of this disclosure may enable surgeons to seamlessly transition between variable clamp configurations during a procedure, thereby enhancing efficiency and adaptability without interrupting the workflow.

The articulating surgical clamp of this disclosure represents a significant advancement over conventional clamps that have fixed jaw configurations that lead to limited functionality. The rigid or fixed-shape nature of currently available clamps forces surgeons to utilize them in a way that may not be optimal for a particular patient's anatomy. The articulating clamp of this disclosure has a jaw portion that includes a series of articulation mechanisms or articulatable segments that can give surgeons precise control and flexibility over the angle, shape, and/or position of the clamp's jaws and can adapt the tool to a particular anatomy as needed.

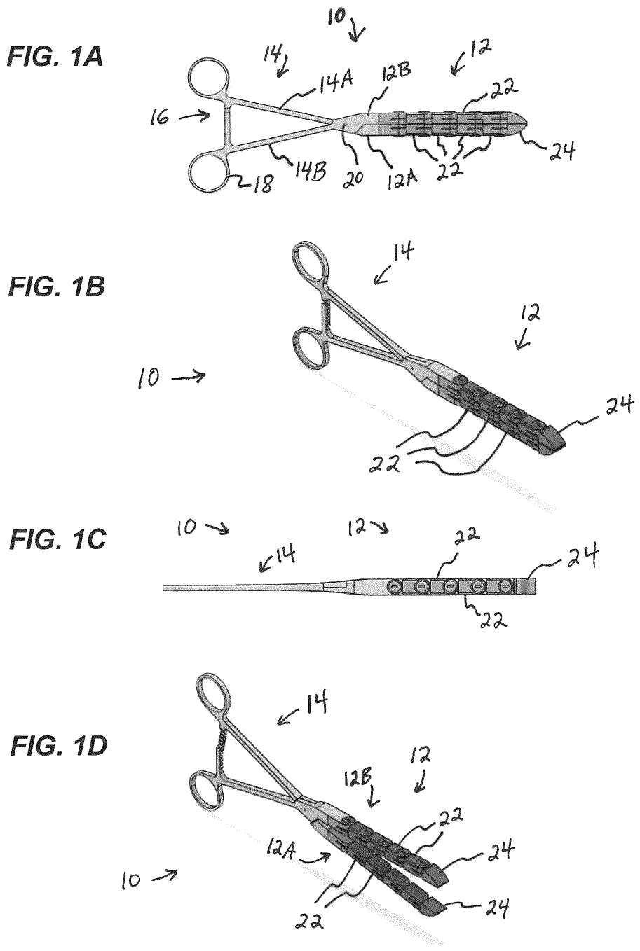

FIG. 1A is a side view of articulating clamp 10 in a closed position according to some embodiments of this disclosure. Clamp 10 includes a handle portion 14 and a jaw portion 12. Clamp 10 may also be described as having two arms pivotably coupled to each other at a pivot joint 20. As such, one pivot arm may have a handle portion 14A and a jaw portion 12A, while the other pivot arm may have a handle portion 14B and a jaw portion 12B, substantially as shown in FIG. 1A. The handle portion 14 may also include features such as finger grips 18 and or a ratcheting mechanism 16, for example. Ratcheting mechanism 16 may, for example, enable a surgeon to position and releasably hold clamp 10 in a desired closed position for vessel closure or sealing applications. Jaw portion 12 may comprise a plurality of articulating segments 22 extending along a length of jaw portion 12, as shown. At a distal end of jaw portion 12, an end portion 24 (or tip portion 24) may be coupled (e.g., articulably coupled) to one of the plurality of articulating segments 22 as shown. It should be noted that clamp 10 may be formed from a variety of biocompatible materials (e.g., stainless steel, titanium, certain polymers, silicon, etc.). For example, in some embodiments, handle portions 14A and 14B may comprise detachable and/or interchangeable composite handle portions 14A and 14B. Such detachable and/or interchangeable composite handle portions 14A and 14B may, for example, be formed of a polymeric material that could be attached to jaw portions 12A and 12B just proximally of hinge 20 (e.g., to the left of hinge 20 as shown in FIG. 1A).

FIG. 1B provides a top perspective view of articulating clamp 10 in the closed position, providing additional details regarding the arrangement and orientation of the articulating segments 22 and tip portion 24 as they relate to the structure of the jaw portion 12 of clamp 10. FIG. 1C provides a top view of articulating clamp 10 in the closed position, illustrating how the articulating segments 22 and tip portion 24 of jaw portion 12 may appear in a substantially straight configuration of clamp 10. FIG. 1D provides a perspective view of articulating clamp 10 with the jaw portion 12 shown in a partially open position while in a straight configuration. As can be seen in FIG. 1D, the individual jaw portions 12A and 12B may each comprise a plurality of articulating segments 22 and tip portions 24.

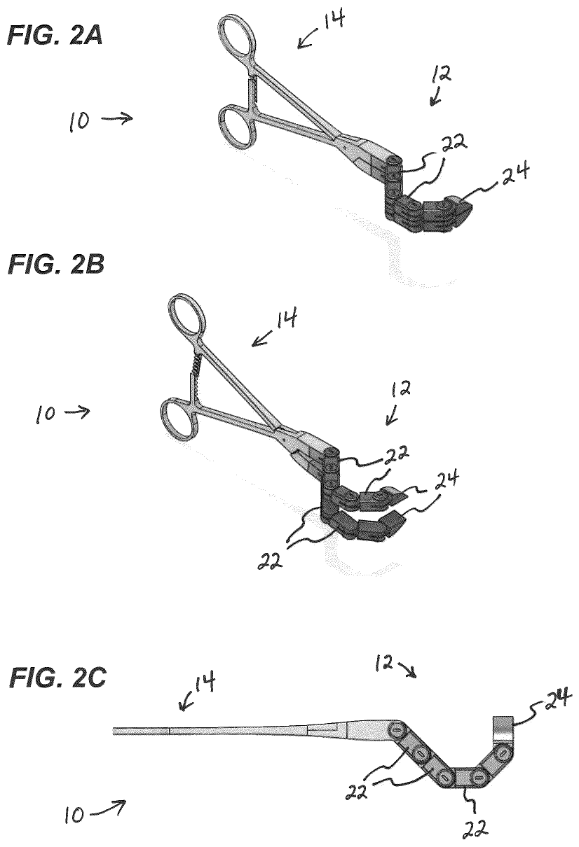

FIG. 2A provides a top perspective view of articulating clamp 10 in the closed position with jaw portion 12 formed into an articulated shape (e.g., curved) according to some embodiments of this disclosure. As shown, the jaw portions 12 of both arms of clamp 10 (e.g., portions 12A and 12B) have been formed into a curved articulated shape as shown. (It should be noted that portion 12A and 12B could be formed into somewhat different shapes from each other, if desired, since the corresponding articulating segments 22 are independently articulable between the two portions 12A and 12B.) As can be seen in FIG. 2A, an angle may be formed between each pair of adjacent articulating segments 22; the particular angles chosen can be selected by a user (e.g., a surgeon) to form the desired overall shape of jaw portion 12 to suit a particular application or anatomy. FIG. 2B provides a top perspective view of articulating clamp 10 with the jaw portion 12 formed in an articulated shape (e.g., a curved shape in the example depicted) and with the handle 14 moved such that the individual arms 12A and 12B of clamp 10 are partially open, as shown. In addition to the articulating segments 22 being articulable with respect to each other, it should also be noted that a first (or most proximal) articulating segment 22 may be articulable with respect to the handle portion 14, and that a last (or most distal) articulating segment 22 may be articulable with respect to the tip portion 24, as shown. This may be more clearly shown in FIG. 2C, which is a top view of articulating clamp 10 in the closed position with jaw portion 12 formed in an articulated shape. In the example shown in FIG. 2C, the first two most proximal articulating segments are shown disposed at roughly a 45-degree angle to the handle portion 14 (with a straight or zero-angle configuration between the first two segments 22). Likewise, the tip portion 24 is shown forming a roughly 45-degree angle with the last (most distal) articulating segment 22. The range of available angles that may be formed at each point of articulation will be discussed in more detail below.

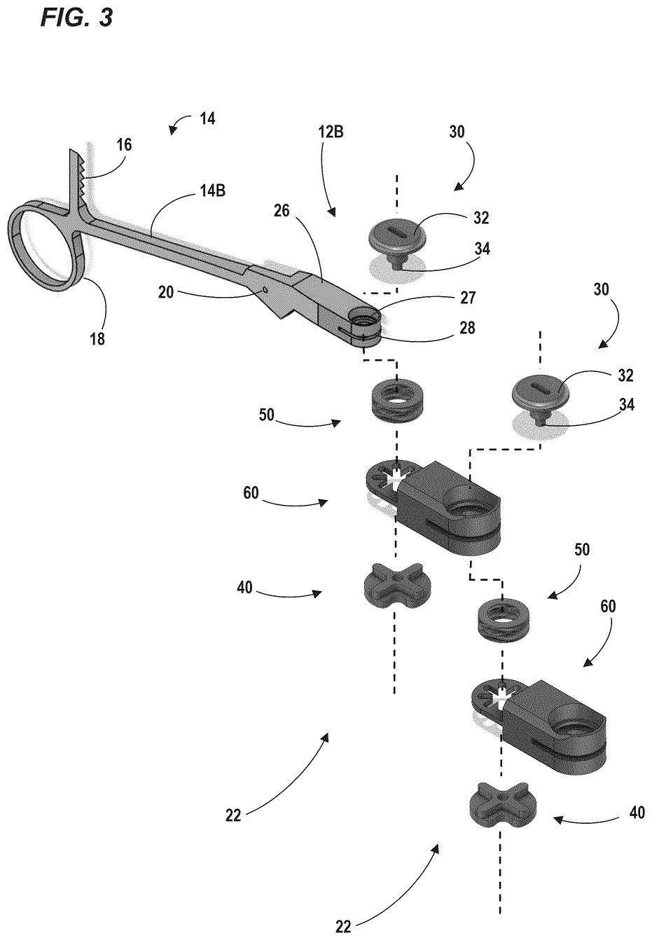

FIG. 3 is an exploded perspective view showing certain portions of articulating clamp 10 according to an embodiment of this disclosure. For example, FIG. 3 shows a portion of one arm of clamp 10, the one arm comprising handle portion 14B and a proximal portion of jaw portion 12B. As shown, jaw portion 12B extends distally from pivot joint 20, and begins with jaw support portion 26. Jaw support portion 26 includes opening 27 and slot 28, which are configured to receive and operably couple with components from the first (most proximal) articulating segment 22, as shown in FIG. 3. In more detail, FIG. 3 illustrates how the first (most proximal) articulating segment 22 operably couples to jaw support portion 26, and how the second (next) articulating segment 22 operably couples to the first segment 22, using similar components in a similar arrangement. The articulating segments 22 of FIG. 3 each include a main body 60, a button 30 (having a button top surface 32 and a lower extension 34), a paddle 40, and a spring 50. These elements interact with each other to form a releasably selectable angular coupling between adjacent main bodies 60, and between a main body 60 and jaw support portion 26, and between a main body 60 and tip portion 24, according to various embodiments of this disclosure. The releasably selectable angle may be formed by rotating a main body 60 about an axis of an opening formed in the main body 60.

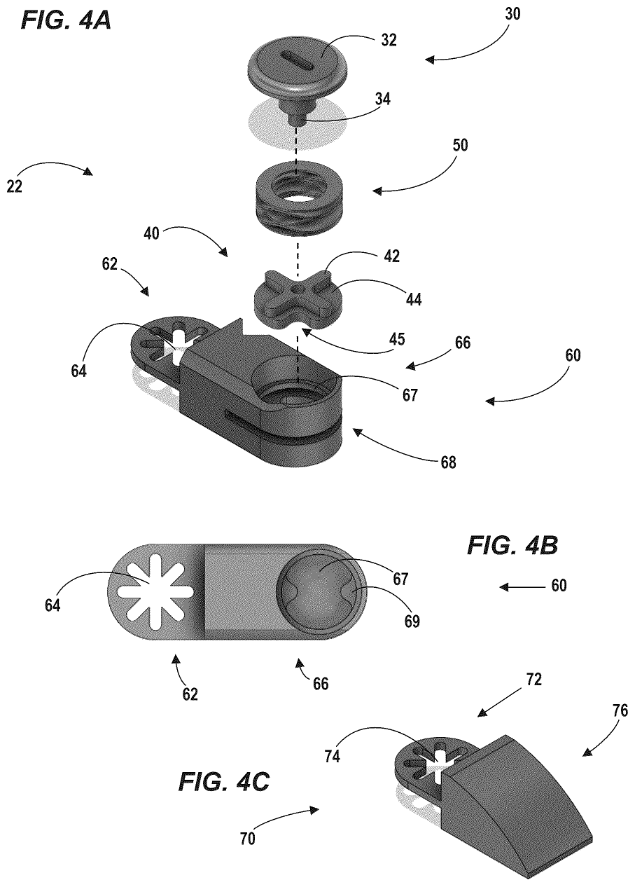

The releasable and selectable nature of the angles that may be formed with respect to each articulating segment 22 will be discussed in more detail with respect to FIGS. 4A and 4B below.

FIG. 4A is an enlarged exploded perspective view of an articulating segment 22 of a jaw portion 12 of an articulating clamp 10 according to certain embodiments of this disclosure. Articulating segment 22 may facilitate angular adjustment to a number of defined angles relative to adjacent segments 22 or other members to thereby enable forming a desired overall shape of the jaw portion 12. This angular adjustment may improve the functionality of the clamp 10 for a particular use, e.g., for a particular anatomical shape for vessel sealing applications, for example. FIG. 4A provides details regarding a number of components that may make up articulating segment 22. A button 30, for example, may be associated with one or more of the plurality of articulating segments 22. Button 30 may have a top surface 32 and a lower extension 34 such that a user may press on top surface 32 of button 30 (e.g., apply a downward or inward force on top surface 32 of button 30), which may cause lower extension 34 to interact with (e.g., move or displace) paddle 40 and thereby enable angular articulation of an adjacent articulating segment 22. In some embodiments, a spring 50 may be employed to provide a bias force against paddle 40; under normal conditions (e.g., no button press), the spring 50 urges the paddle 40 into engagement with an opening 64 in a portion 62 of an adjacent articulating segment 22, while during a sufficient button press event, the bias force of spring 50 is overcome and paddle 40 is thereby disengaged from opening 64 of the adjacent segment 22, allowing the adjacent segment 22 to be angularly adjusted (rotated) relative to another articulating segment 22 (or to jaw support portion 26, or to tip portion 24, etc.).

As can be further seen in FIG. 4A, the angular adjustments that may be formed between adjacent members may comprise a number of defined angles determined by the structure of some of the components. For example, portion 62 extending from main body 60 of an articulating segment 22 may include an opening 64 that comprises a “star” shape or an “asterisk” shape. The shape of opening 64 may facilitate engagement or seating of the paddle 40 into a number of possible defined (“detent”) angular positions. In the example depicted in FIG. 4A, the shaped opening 64 may comprise a pattern with “arms” of the opening 64 extending outwardly at 45-degree increments from the center of the star- or asterisk-shaped opening. Paddle 40 is shown with four arms 42 extending upwardly from a base portion 44, and outwardly at right angles (90 degrees) such that, when disengaged from opening 64, it may be rotated in 45-degree increments to re-engage in a number of different angular positions within the shaped opening 64 of an adjacent segment's main body 60. This may, for example, enable positioning an adjacent articulating segment 22 at one of five (or more) possible angular orientations: 90, 45, 0, −45, and −90 degrees, for example, relative to each other. Of course, other angular increments are contemplated by this disclosure and may be used to achieve different levels of angular customization. For example, 30-degree increments might be used instead, which could enable 7 or more possible angular adjustments or orientations between adjacent segments: 90, 60, 30, 0, −30, −60, and −90 degrees, for example, relative to each other. Fewer or greater numbers of angular adjustments may be accomplished if and as needed.

With continued reference to FIG. 4A, main body 60 of articulating segment 22 has portion 62 (described above with respect to shaped opening 64) and portion 66, which has a recess 67 and a slot 68, as shown. Recess 67 is sized and shaped to receive button 30, paddle 40, and spring 50 therewithin. Slot 68 intersects recess 67 and is sized and shaped to receive a portion 62 of an adjacent articulating segment 22 (or jaw support portion 26, or tip portion 24). Portion 62 extends from main body 60 and is sized and shaped to be slidably inserted into slot 68 such that opening 64 and recess 67 are substantially axially aligned with each other. In some embodiments, recess 67 may include one or more protuberances 69 (shown in FIG. 4B) configured to align with corresponding indentations 45 formed in a perimeter of base portion 44 of paddle 40. The alignment of indentations 45 and protuberances 69 function to keep paddle 40 in a fixed angular relationship within recess 67. Accordingly, it is the angular rotation of the shaped opening 64 of an adjacent segment 22 that can be selectively engaged and disengaged with the arms 42 of paddle 40 to facilitate angular adjustments. FIG. 4B is an enlarged top view of main body 60 of an articulatable segment 22, showing portion 62 (having the shaped opening 64) and portion 66 (having recess 67 and, in some embodiments, one or more protuberances 69).

FIG. 4C is an enlarged perspective view of an end body 70 of an articulatable tip portion 24 according to some embodiments of this disclosure. End body 70 includes elements and features similar to those of main body 60 described above, but does not have a recess 67. For example, end body has portion 72 with a shaped opening 74, and a portion 76 that forms an end piece, which may be tapered or smooth or shaped as deemed necessary, according to various embodiments of this disclosure.

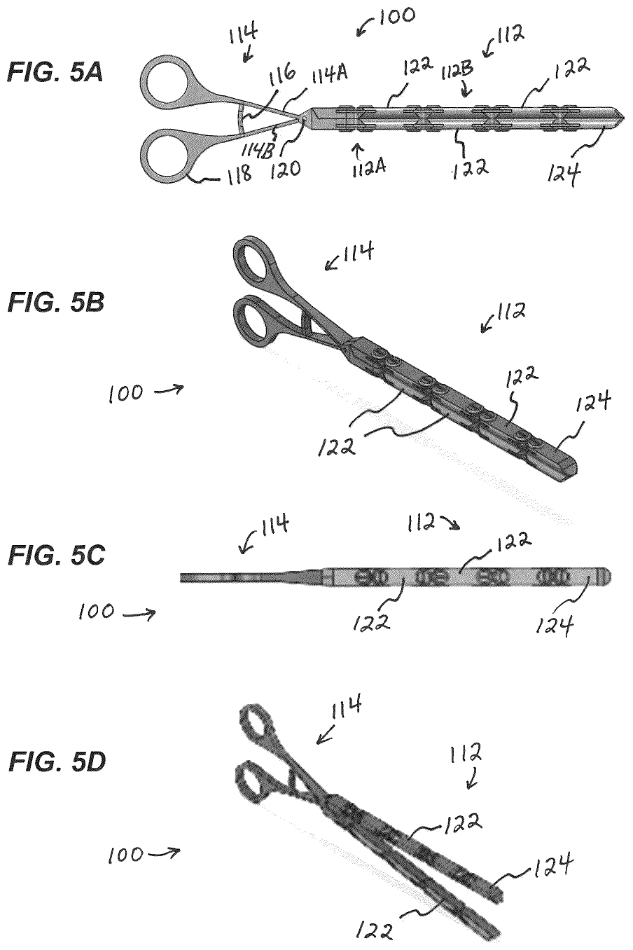

FIG. 5A is a side view of articulating clamp 10 according to some embodiments of this disclosure. Clamp 100 includes a handle portion 114 and a jaw portion 112. Clamp 100 may also be described as having two arms pivotably coupled to each other at a pivot joint 120. As such, one pivot arm may have a handle portion 114A and a jaw portion 112A, while the other pivot arm may have a handle portion 114B and a jaw portion 112B, substantially as shown in FIG. 5A. The handle portion 114 may also include features such as finger grips 118 and or a ratcheting mechanism 116, for example. Ratcheting mechanism 116 may, for example, enable a surgeon to position and releasably hold clamp 100 in a desired closed position for vessel closure or ceiling applications. Jaw portion 112 may comprise a plurality of articulating segments 122 extending along a length of jaw portion 112, as shown. At a distal end of jaw portion 112, an end portion 124 (or tip portion 124) may be coupled (e.g., articulably coupled) to one of the plurality of articulating segments 122 as shown. FIG. 5B provides a top perspective view of articulating clamp 100, providing additional details regarding the arrangement and orientation of the articulating segments 122 and tip portion 124 as they relate to the structure of the jaw portion 112 of clamp 100. FIG. 5C provides a top view of articulating clamp 100 illustrating how the articulating segments 122 and tip portion 124 of jaw portion 112 may appear in a substantially straight configuration of clamp 100. FIG. 5D provides a perspective view of articulating clamp 100 with the jaw portion 112 shown in a partially open position while in a straight configuration. As can be seen in FIG. 5D, the individual jaw portions 112A and 112B may each comprise a plurality of articulating segments 122 and tip portions 124.

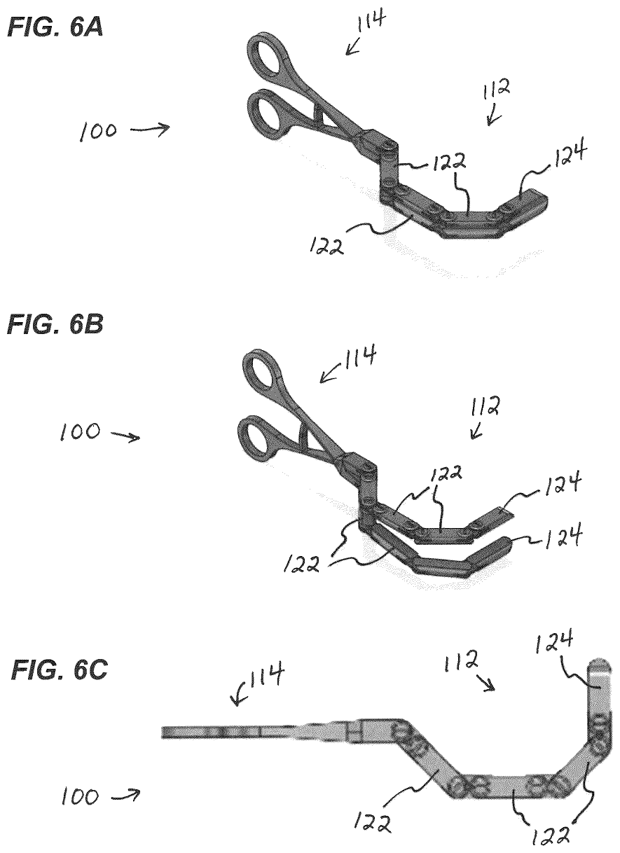

FIG. 6A provides a top perspective view of articulating clamp 100 with jaw portion 112 formed into an articulated shape (e.g., curved) according to some embodiments of this disclosure. As shown, the jaw portions 112 of both arms of clamp 100 (e.g., portions 112A and 112B) have been formed into a curved articulated shape as shown. (It should be noted that portions 112A and 112B could be formed into somewhat different shapes from each other, if desired, since the corresponding articulating segments 122 are independently articulable between the two portions 112A and 112B.) As can be seen in FIG. 6A, an angle may be formed between each pair of adjacent articulating segments 122; the particular angles chosen can be selected by a user (e.g., a surgeon) to form the desired overall shape of jaw portion 112 to suit a particular application or anatomy. FIG. 6B provides a top perspective view of articulating clamp 100 with the jaw portion 112 formed in an articulated shape (e.g., a curved shape in the example depicted) and with the handle 114 moved such that the individual arms 112A and 112B of clamp 100 are partially open, as shown. In addition to the articulating segments 122 being articulable with respect to each other, it should also be noted that a first (or most proximal) articulating segment 122 may be articulable with respect to the handle portion 114, and that a last (or most distal) articulating segment 122 may be articulable with respect to the tip portion 124, as shown. This may be more clearly shown in FIG. 6C, which is a top view of articulating clamp 100 with jaw portion 112 formed in an articulated shape. In the example shown in FIG. 6C, the first two most proximal articulating segments are shown disposed at roughly a 45-degree angle to the handle portion 114. Likewise, the tip portion 124 is shown forming a roughly 45-degree angle with the last (most distal) articulating segment 122. The range of available angles that may be formed at each point of articulation will be discussed in more detail below.

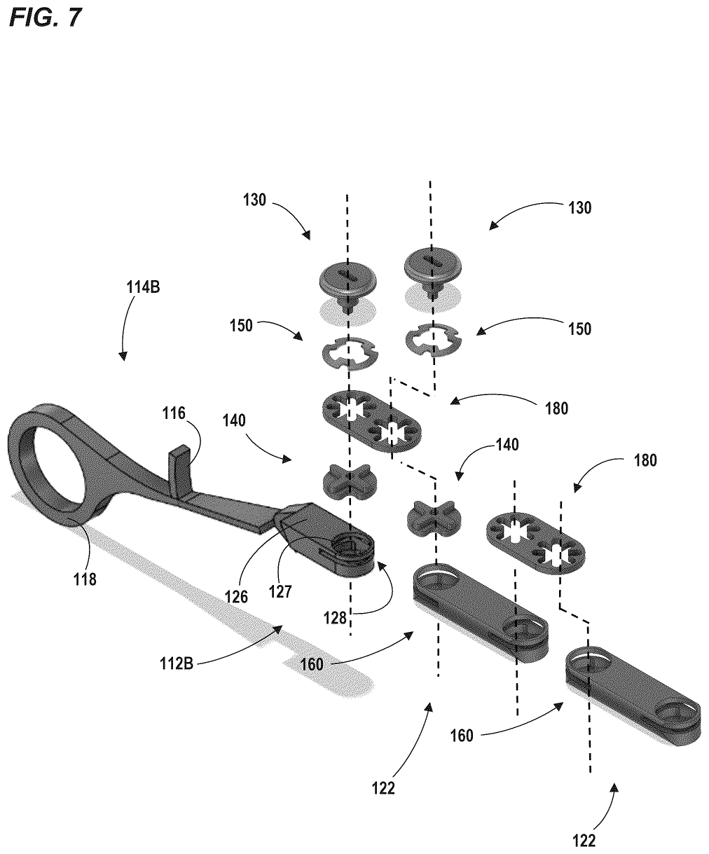

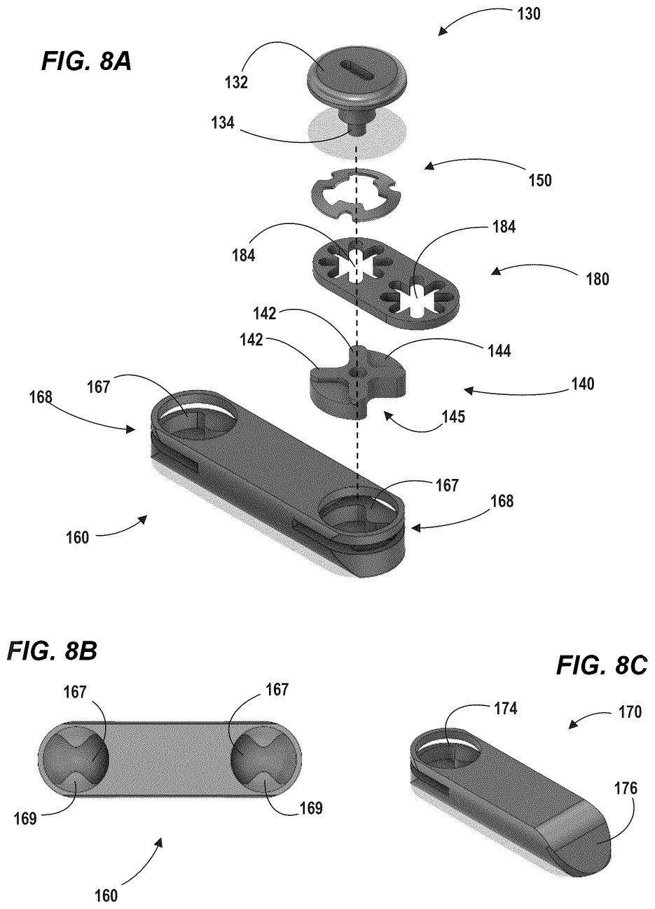

FIG. 7 is an exploded perspective view showing certain portions of articulating clamp 100 according to an embodiment of this disclosure. For example, FIG. 7 shows a portion of one arm of clamp 100, the one arm comprising handle portion 114B and a proximal portion of jaw portion 112B. As shown, jaw portion 112B extends distally from handle portion 114B, and includes a jaw support portion 126. Jaw support portion 126 includes opening or recess 127 and slot 128, which are configured to receive and operably couple with components associated with the first (most proximal) articulating segment 122, as shown in FIG. 7. In more detail, FIG. 7 illustrates how the first (most proximal) articulating segment 122 operably couples to jaw support portion 126, and how the second (next) articulating segment 122 operably couples to the first segment 122, using similar components in a similar arrangement. The articulating segments 122 of FIG. 7 each include a main body 160, a pair of buttons 130 (each having a button top surface 132 and a lower extension 134, shown in FIG. 8A), a pair of paddles 140, and a pair of springs 150. FIG. 7 also depicts a linkage 180 configured to extend between and operably couple articulating segments 122 to each other, as well as to the jaw support portion 126 and the tip portion 124. These elements interact with each other to form a releasably selectable angular coupling at the points where the main bodies 160 couple to a linkage 180, according to various embodiments of this disclosure. The releasably selectable angle may be formed by rotating a main body 160 about an axis of an opening or recess formed in the main body 160 and in an associated linkage 180. The releasable and selectable nature of the angles that may be formed with respect to each articulating segment 122 will be discussed in more detail with respect to FIGS. 8A and 8B below.

FIG. 8A is an enlarged exploded perspective view of an articulating segment 122. FIG. 8A provides details regarding a number of components that may make up articulating segment 122, according to some embodiments. A button 130, for example, may be associated with one or more of the plurality of articulating segments 122. Button 130 may have a top surface 132 and a lower extension 134 such that a user may press on top surface 132 of button 130 (e.g., apply a downward or inward force on top surface 132 of button 130), which may cause lower extension 134 to interact with (e.g., move or displace) paddle 140 and thereby enable angular articulation relative to a linkage 180 coupled to an adjacent articulating segment 122. In some embodiments, a spring 150 may be employed to provide a bias force against paddle 140; under normal conditions (e.g., with no button press), the spring 150 urges the paddle 140 into engagement with an opening 184 in a linkage, while during a sufficient button press event, the bias force of spring 150 is overcome and paddle 140 is thereby disengaged from opening 184 of the linkage 180, allowing an angular adjustment (rotation) to occur between main body 160 of articulating segment 122 and the affected linkage 180 (or between a jaw support portion 126 and a linkage 180, or between a tip portion 124 and a linkage 180, etc.).

As can be further seen in FIG. 8A, the angular adjustments that may be formed between articulating segments 122 and linkages 180 may comprise a number of defined angles determined by the structure of some of the components. For example, linkage 180 may include a pair of openings 184, each of which may comprise a “star” shape or an “asterisk” shape. The shape of opening 184 may facilitate engagement or seating of a respective paddle 140 into a number of possible defined (“detent”) angular positions within said opening 184. In the example depicted in FIG. 8A, the shaped opening 184 may comprise a pattern with “arms” of the opening 184 extending outwardly at 45-degree increments from the center of the star- or asterisk-shaped opening. Paddle 140 is shown with four arms 142 extending upwardly from a base portion 144, and outwardly at right angles (90 degrees) such that, when disengaged from opening 184, it may be rotated in 45-degree increments to re-engage in a number of different angular positions within the shaped opening 184 of an adjacent linkage 180. This may, for example, enable positioning an articulating segment 122 at one of five (or more) possible angular orientations relative to an adjacent linkage 180: for example, 90, 45, 0, −45, and −90 degree angles relative to each other. Of course, other angular increments are contemplated by this disclosure and may be used to achieve different levels of angular customization. For example, 30-degree increments might be used instead, which could enable 7 or more possible angular adjustments or orientations between adjacent segments and/or linkages: 90, 60, 30, 0, −30, −60, and −90 degrees, for example, relative to each other. Fewer or greater numbers of angular adjustments may be accomplished if and as needed.

With continued reference to FIG. 8A, main body 160 of articulating segment 122 has a pair of recesses 167 and a pair of slots 68, disposed at either end of main body 160 as shown. Each recess 167 is sized and shaped to receive button 130, paddle 140, and spring 150 therewithin. Slots 168 intersect each recess 167 and are sized and shaped to receive a corresponding portion of a linkage 180. Each linkage 180 is sized and shaped to be slidably inserted into a slot 168 such that one of the openings 184 of linkage and the recess 167 are substantially axially aligned with each other. In some embodiments, recesses 167 may each include one or more protuberances 169 (shown in FIG. 8B) configured to align with corresponding indentations 145 formed in a perimeter of base portion 144 of paddle 140. The alignment of indentations 145 and protuberances 169 function to keep paddle 140 in a fixed angular relationship within recess 167. Accordingly, it is the angular rotation of the shaped opening 184 of an adjacent linkage 180 that can be selectively engaged and disengaged with the arms 142 of paddle 140 to facilitate angular adjustments. FIG. 8B is an enlarged top view of main body 160 of an articulatable segment 122, showing an exemplary arrangement of recesses 167 and, in some embodiments, one or more protuberances 169 within a recess 167.

FIG. 8C is an enlarged perspective view of an end body 170 of an articulatable tip portion 124 according to some embodiments. End body 170 includes a recess 174 and a portion 176 that forms an end piece, which may be tapered or smooth or shaped as deemed necessary, according to various embodiments of this disclosure. Recess 174 is configured to couple with a linkage 180 in a manner similar to that described above with respect to the articulating segments 122.

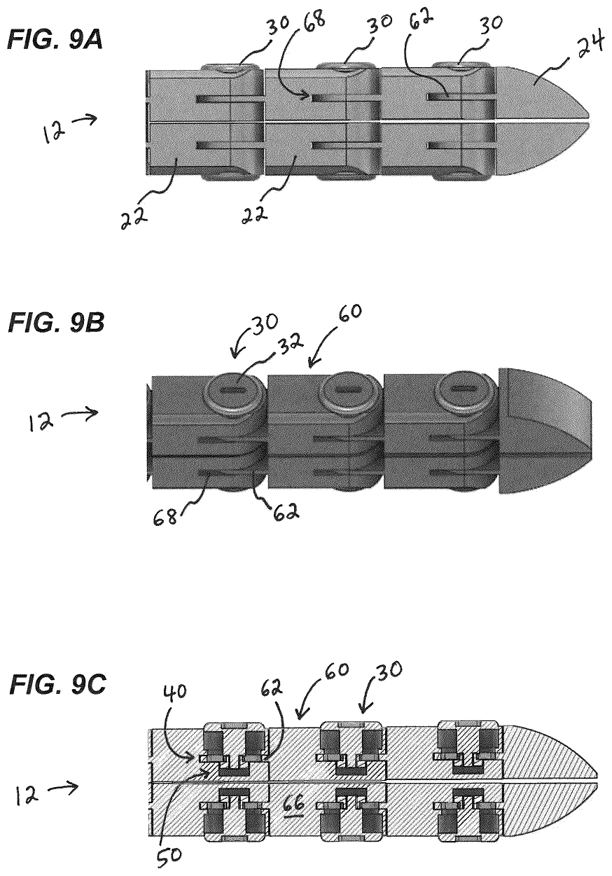

FIGS. 9A-9C are enlarged images showing additional details regarding the arrangement of components associated with the articulating segments 22 of jaw portion 12. FIG. 9A is an enlarged top view of jaw portion 12, showing the alignment of buttons 30 and how portions 62 of one segment 22 extend into a slot 68 of an adjacent segment 22, for example. FIG. 9B is an enlarged top oblique/rotated view of a jaw portion 12, showing additional details regarding the arrangement and alignment of the elements discussed with respect to FIG. 9A. FIG. 9C is an enlarged cross-sectional view of a jaw portion 12 of an articulating clamp 10 according to some embodiments of this disclosure. An exemplary arrangement of components is depicted in FIG. 9C, including paddle 40 and spring 50, and their positions relative to button 30 and portion 62.

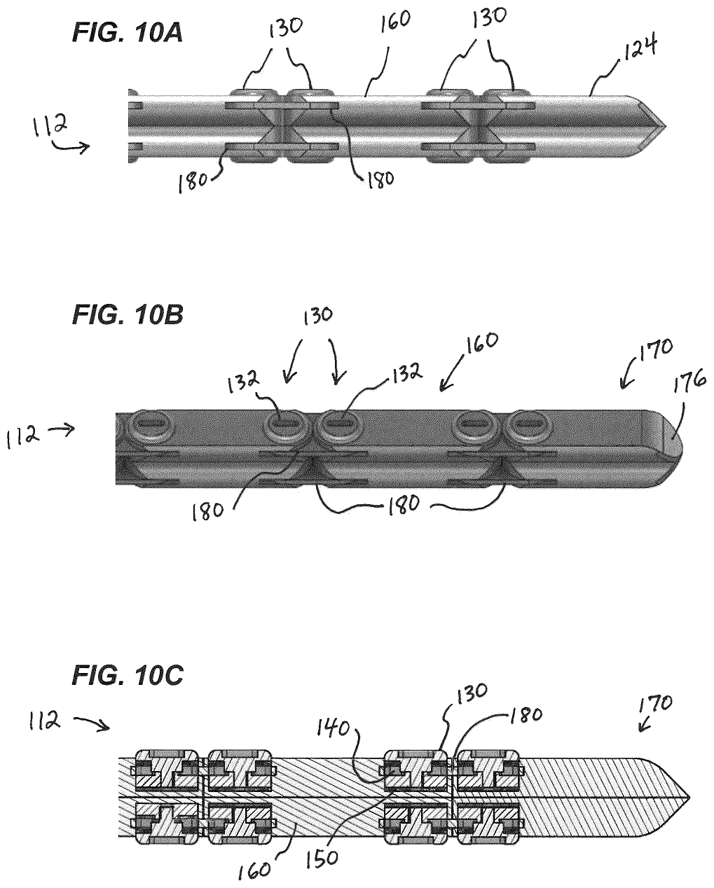

FIGS. 10A-10C are enlarged images showing additional details regarding the arrangement of components associated with the articulating segments 122 of jaw portion 112. FIG. 10A is an enlarged top view of jaw portion 112, showing the alignment of buttons 130 and how linkages 180 extend into slots 168 of one or more adjacent segments 122, for example. FIG. 10B is an enlarged top oblique/rotated view of a jaw portion 112, showing additional details regarding the arrangement and alignment of the elements discussed with respect to FIG. 10A. FIG. 10C is an enlarged cross-sectional view of a jaw portion 112 of an articulating clamp 100 according to some embodiments of this disclosure. An exemplary arrangement of components is depicted in FIG. 10C, including paddle 140 and spring 150, and their positions relative to buttons 130 and linkages 180.

Some embodiments of this disclosure relate to a surgical clamp that allows a handle portion to be releasably or detachably engaged to a jaw portion, allowing a number of different jaw portion designs to be securely attached to a number of different handle designs. This modular feature gives users the flexibility to customize their instruments to meet the specific needs of the procedure and patient, while maintaining operational safety, case of use, and precise functionality. The jaw portion, for example, may be selected for use among various jaw profiles (such as straight, curved, or angled jaws) and different articulation features or mechanisms depending on the application (e.g., selected for specific surgical environments or tissue types). Likewise, the range of handle variants available may differ in terms of ergonomics (e.g., different shapes, lengths, or textures to accommodate user preferences), grip style, functional features (e.g., handles with integrated feedback mechanisms), and/or mechanical properties, enabling a selection that may enhance the utility of the resulting device and/or the level of comfort to the user.

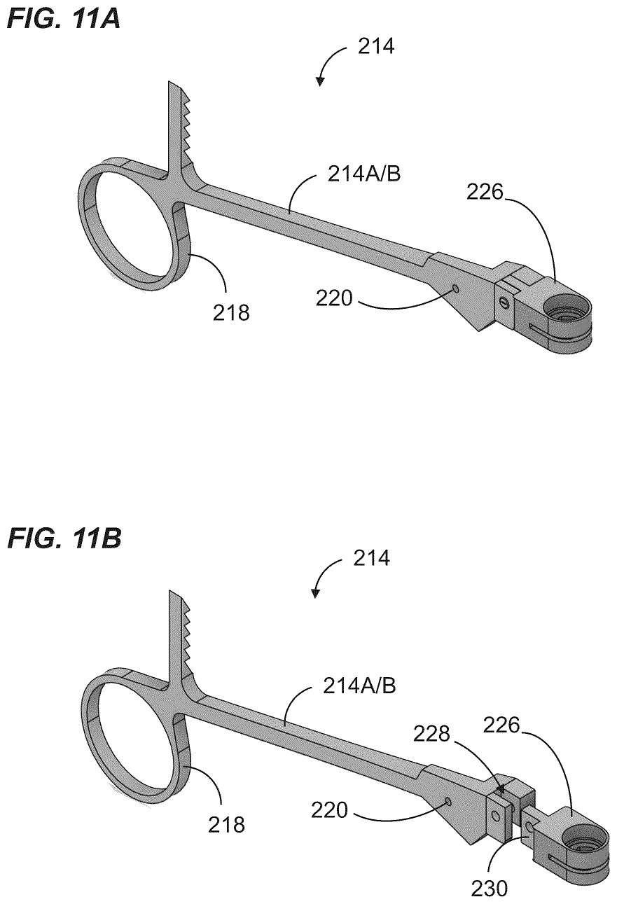

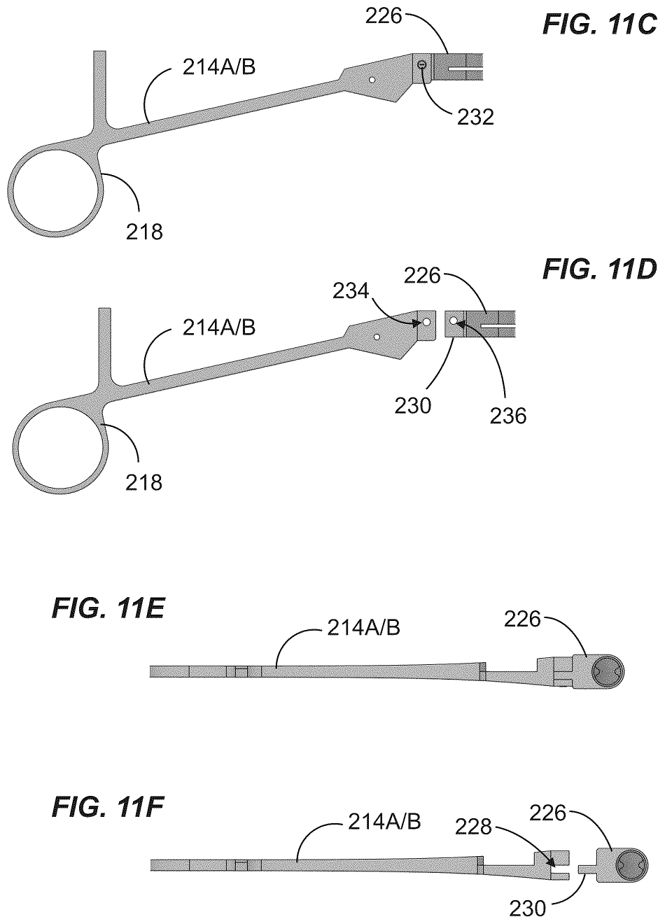

FIG. 11A is perspective view showing certain portions of an articulating clamp according to some embodiments of this disclosure. For example, FIG. 11A shows a portion of one arm of handle portion 214 of an articulating clamp, the one arm comprising a detachable handle portion 214B. As noted above, in some embodiments, an exemplary handle portion 214A/B may comprise a detachable and/or interchangeable handle portion 214A/B. (Note: A corresponding second handle portion is not shown in FIG. 11A but would be substantially similar to form a functional pair of handle portions. For example, handle portions 214A and 214B may be pivotably coupled to one another about a pivot joint 220, as shown in FIG. 11A.) A pair of such detachable and/or interchangeable composite handle portions 214A/B may, for example, be formed of a polymeric material that could be attached to a jaw portion via a jaw support portion 226 as shown in FIG. 11A.

FIG. 11B is an exploded perspective view showing portions of the articulating clamp of FIG. 11A according to some embodiments of this disclosure. For example, FIG. 11B shows a slot 228 formed in a distal end of handle portion 214A/B. Slot 228 may be configured to receive a complementary tab portion 230 of jaw support portion 226 therewithin. Slot 228 may be configured to be releasably and/or detachably coupled to the complementary tab portion 230 of jaw support portion 226 as shown. Of course, other comparable configurations are contemplated. For example, a slot may be formed in a proximal portion of jaw support portion 226, and a complementary tab may extend from a distal end of handle portion 214A/B, according to some embodiments. Other releasably detachable coupling mechanisms between the handle portion 214A/B and the jaw support portion 226 are contemplated and are deemed to be within the scope of this disclosure.

FIG. 11C is a side view showing portions of the articulating clamp of FIG. 11A, according to some embodiments of this disclosure. FIG. 11C shows an exemplary coupling mechanism 232 that may be configured to provide a releasable engagement (e.g., a detachable connection or an interchangeable connection) between the handle portion 214A/B and the jaw support portion 226 (and to any jaw portions coupled thereto) and is constructed to allow for quick and secure attachment and detachment between the handle portion 214A/B and the jaw support portion 226. Coupling mechanism 232 may, for example, include a pivot pin 232 that is positioned through holes 234 and 236 formed in the tab 236 and/or adjacent the slot 228, according to some embodiments. Such a configuration of coupling mechanism 232 may help ensure that a reliable mechanical attachment is formed between the handle portion 214A/B and the jaw support portion 226. In some cases, the coupling mechanism may further comprise a spring-loading mechanism to facilitate the releasable engagement. Additionally, or optionally, a release mechanism, such as a pushbutton, may be used to facilitate the releasable engagement of the handle portion 214A/B with the jaw support portion 226, either with or without a spring-loaded mechanism.

FIG. 11D is an exploded side view showing portions of the exemplary articulating clamp of FIG. 11A, according to some embodiments of this disclosure. FIG. 11D shows an example of suitable positioning of holes 234 and 236 formed in tab 236 and adjacent the slot 228, according to some embodiments. In some embodiments, having a complementary shape (e.g., a rectangular shaped tab 230 configured to engage within a similar rectangular shaped slot 228 may help form a more secure engagement between the handle portion 214A/B and the jaw support portion 226 when coupled together.

The coupling mechanism 232 between the handle portion 214A/B and the jaw support portion 226 may be configured or constructed to allow for quick and secure attachment and detachment of these respective components. Coupling mechanism 232 may include a connector interface to facilitate modular coupling of a range of handle portions 214A/B and/or jaw support portions 226. The connector interface may be configured to allow any variant of handle portion 214A/B to be securely attached to any variant of jaw support portion 226. In some embodiments, the connector interface may include a male/female coupling system (e.g., such as the pivot pin 232 positioned through holes 234 and 236 in tab 236 and/or slot 228, as described above with respect to FIG. 11C). In other embodiments, the connector interface may include other types of releasable engagement mechanisms, including without limitation, a threaded twist-lock mechanism, a friction fit, a collet and release arrangement, etc. In some embodiments, the connector interface may also include a positive locking mechanism (e.g., such as a spring-loaded latch or a push-button release) that ensures that the handle portion 214A/B and the jaw support portion 226 remain securely attached during the procedure and can only be detached through an intentional release mechanism.

The coupling mechanism 232 may be designed to withstand typical operational conditions (e.g., torsional and compressive forces exerted upon the clamp during clamping and articulation without failure or unintended disconnection). Materials used in the interface may be selected to provide a high level of durability, sterilizability, or mechanical strength, or any combination of these parameters, including for example, surgical-grade stainless steel or titanium.

The coupling mechanism 232 may also help to ensure that, when coupled together, the handle portion 214A/B and the jaw support portion 226 are securely fastened so that force transmission from the handle portion 214A/B to the jaw support portion 226 (and to jaw portions coupled thereto) is efficient and/or consistent (e.g., to provide for more precise control of the articulating jaws during delicate surgical procedures).

FIG. 11E is a top view, and FIG. 11F is an exploded top view, showing portions of the articulating clamp of FIG. 11A, according to some embodiments of this disclosure. For example, FIGS. 11E and 11F show an exemplary configuration with an alignment of the tab 230 of jaw support portion 226 within the slot 228 of handle portion 214A/B, according to some embodiments of this disclosure. The sizing and shape of the tab 230 and the slot 228, as mentioned above, may facilitate forming a secure engagement or coupling between the handle portion 214A/B and the jaw support portion 226, which may improve the performance characteristics of the articulating clamp during use.

While the various systems described above are generally described as distinct implementations, any of the individual components, mechanisms, or devices, and related features and functionality, within the various embodiments described in detail above can be incorporated into any of the other embodiments described herein. For example, while two different types of articulatable segments (for forming the respective jaw portions) are described with reference to two separate surgical clamp embodiments, it is contemplated herein that both types of articulatable segments could be employed within a single clamp design, as would be apparent to one of ordinary skill in the art with the benefit of these teachings.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A surgical clamp, the surgical clamp comprising:
two pivot arms pivotably coupled to each other via a hinge, each of the pivot arms having a handle portion disposed proximal to the hinge and a jaw portion disposed distal to the hinge, the jaw portion of each pivot arm comprising:
a plurality of articulable segments, each of the plurality of articulable segments being pivotably coupled to at least one adjacent articulable segment to enable providing the jaw portion with an adjustable shape.

2. The surgical clamp of claim 1, wherein each of the plurality of articulable segments has a first end and a second end, and wherein the plurality of articulable segments are pivotably coupled to each other in an end-to-end configuration.

3. The surgical clamp of claim 1, wherein each of the plurality of articulable segments can be pivoted to an angle with respect to an adjacent articulable segment and locked at the angle with respect to the adjacent articulable segment.

4. The surgical clamp of claim 3, wherein each of the plurality of articulable segments is configured to be pivoted to and locked at a finite number of discrete angles with respect to the adjacent articulable segment.

5. The surgical clamp of claim 1, wherein the handle portion and the jaw portion of at least one of the two pivot arms are releasably coupled to each other.

6. The surgical clamp of claim 5, wherein the releasable coupling between the handle portion and the jaw portion facilitates replacing the handle portion with a second handle portion.

7. The surgical clamp of claim 5, wherein the releasable coupling between the handle portion and the jaw portion facilitates replacing the jaw portion with a second jaw portion.

8. The surgical clamp of claim 5, wherein the releasable coupling between the handle portion and the jaw portion comprises a tab extending proximally from the jaw portion and a slot facing distally from the handle portion.

9. The surgical clamp of claim 5, wherein the releasable coupling between the handle portion and the jaw portion comprises a tab extending distally from the handle portion and a slot facing proximally from the jaw portion.

10. The surgical clamp of claim 8, wherein tab and the slot are sized and shaped in a complementary manner to facilitate a secure engagement between the handle portion and the jaw portion when coupled together.

11. The surgical clamp of claim 8, further comprising a pin configured to engage through a first opening in the tab and a second opening in the slot.

12. The surgical clamp of claim 11, wherein the pin is biased by spring tension and the releasable coupling between the handle portion and the jaw portion is actuatable by a button coupled to the spring.

13. A surgical clamp, the surgical clamp comprising:
two pivot arms pivotably coupled to each other via a hinge, each of the pivot arms having a handle portion disposed proximal to the hinge and a jaw portion disposed distal to the hinge,
wherein the jaw portion of each pivot arm comprises:
a plurality of articulable segments, each of the plurality of articulable segments being pivotably coupled to at least one adjacent articulable segment to enable providing the jaw portion with an adjustable shape; and
wherein the handle portion and the jaw portion of the two pivot arms are releasably coupled to each other.

14. The surgical clamp of claim 13, wherein the releasable coupling between the handle portion and the jaw portion comprises:
a tab extending from one of the handle portion and the jaw portion; and
a slot extending from the other of the handle portion and the jaw portion.

15. The surgical clamp of claim 14, wherein tab and the slot are sized and shaped in a complementary manner to facilitate a secure engagement between the handle portion and the jaw portion when coupled together.

16. The surgical clamp of claim 15, further comprising a pin configured to engage through a first opening in the tab and a second opening in the slot.

17. The surgical clamp of claim 16, wherein the pin is biased by spring tension and the releasable coupling between the handle portion and the jaw portion is actuatable by a button coupled to the spring.

18. A surgical clamp, the surgical clamp comprising:
two pivot arms pivotably coupled to each other via a hinge, each of the pivot arms having a handle portion disposed proximal to the hinge and a jaw portion disposed distal to the hinge, the jaw portion of each pivot arm comprising:
a plurality of articulable segments, each of the plurality of articulable segments being pivotably coupled to at least one adjacent articulable segment to enable providing the jaw portion with an adjustable shape,
wherein each of the plurality of articulable segments comprises:
a main body, the main body comprising:
a first end, a coupling portion extending outwardly from the first end of the main body, the coupling portion having a shaped opening formed therein; and
a second end having a recess formed therein and a slot formed therein, the slot intersecting the recess, the slot configured to receive a coupling portion of an adjacent articulating segment, and
a releasable engagement mechanism configured to selectively engage the coupling portion of the adjacent articulating segment at a desired angle with respect to the articulating segment.

19. The surgical clamp of claim 18, wherein the releasable engagement mechanism comprises a pushbutton and a paddle disposed within the recess, the pushbutton configured to move the paddle into and out of selective engagement with the coupling portion of the adjacent articulating segment positioned within the slot of the articulating segment.

20. The surgical clamp of claim 19, wherein the releasable engagement mechanism further comprises a spring to bias a position of the paddle within the recess.
